SEQUENCE LISTING (60 sequences)

SEQ ID NO: 1 - DNA, 20 bp, Artificial Sequence
  Synthetic Construct
cccctaccatgactttattc

SEQ ID NO: 2 - DNA, 20 bp, Artificial Sequence
  Synthetic Construct
attgcactcatcagagctac

SEQ ID NO: 3 - DNA, 20 bp, Artificial Sequence
  Synthetic Construct
agtcatggtaggggagcttg

SEQ ID NO: 4 - DNA, 36 bp, Artificial Sequence
  Synthetic Construct
agucaugguaggggagcuugguuuuagagcuaugcu

SEQ ID NO: 5 - DNA, 36 bp, Artificial Sequence
  Synthetic Construct
auugcacucaucagagcuacguuuuagagcuaugcu

SEQ ID NO: 6 - DNA, 36 bp, Artificial Sequence; identical to SEQ ID NO: 4
  Synthetic Construct

SEQ ID NO: 7 - DNA, 22 bp, Artificial Sequence
  Synthetic Construct
gtctgctccaggtgatgtttat

SEQ ID NO: 8 - DNA, 21 bp, Artificial Sequence
  Synthetic Construct
gggcctgagaatctgcattta

SEQ ID NO: 9 - DNA, 20 bp, Artificial Sequence
  Synthetic Construct
ggggccactagggacaggat

SEQ ID NO: 10 - DNA, 20 bp, Artificial Sequence
  Synthetic Construct
ctgaactcgcagttggccat

SEQ ID NO: 11 - DNA, 20 bp, Artificial Sequence
  Synthetic Construct
ctgaactcgcagttggccat

SEQ ID NO: 12 - DNA, 20 bp, Artificial Sequence
  Synthetic Construct
ctgtgcgtgcagttggccat

SEQ ID NO: 13 - DNA, 20 bp, Artificial Sequence
  Synthetic Construct
gttaacttacagttggccat

SEQ ID NO: 14 - DNA, 20 bp, Artificial Sequence
  Synthetic Construct
tggacctctcagttggccat

SEQ ID NO: 15 - DNA, 20 bp, Artificial Sequence
  Synthetic Construct
ctgaacactgagttggccat

SEQ ID NO: 16 - DNA, 20 bp, Artificial Sequence
  Synthetic Construct
ttgaacttgtagttggccaa

SEQ ID NO: 17 - DNA, 20 bp, Artificial Sequence
  Synthetic Construct
ccgacctggcagttggccct

SEQ ID NO: 18 - DNA, 20 bp, Artificial Sequence
  Synthetic Construct
cccacctcgcaggtggccat

SEQ ID NO: 19 - DNA, 20 bp, Artificial Sequence
  Synthetic Construct
cccacctcgcaggtggccat

SEQ ID NO: 20 - DNA, 20 bp, Artificial Sequence
  Synthetic Construct
ctcatctggcagttggcctt

SEQ ID NO: 21 - DNA, 20 bp, Artificial Sequence
  Synthetic Construct
cctaactcccagttggccag

SEQ ID NO: 22 - DNA, 20 bp, Artificial Sequence
  Synthetic Construct
ctgtcttctcagttggccat

SEQ ID NO: 23 - DNA, 20 bp, Artificial Sequence
  Synthetic Construct
cagaaatggcagttggccag

SEQ ID NO: 24 - DNA, 20 bp, Artificial Sequence
  Synthetic Construct
atggactcacatttggccat

SEQ ID NO: 25 - DNA, 20 bp, Artificial Sequence
  Synthetic Construct
cttcactcccagttggtcat

SEQ ID NO: 26 - DNA, 20 bp, Artificial Sequence
  Synthetic Construct
ctggactcctatttggccat

SEQ ID NO: 27 - DNA, 20 bp, Artificial Sequence
  Synthetic Construct
ctcaacgtgcagctggccat

SEQ ID NO: 28 - DNA, 20 bp, Artificial Sequence
  Synthetic Construct
ttggactcgctgttggcctt

SEQ ID NO: 29 - DNA, 20 bp, Artificial Sequence
  Synthetic Construct
ttgaactggctggtggccat

SEQ ID NO: 30 - DNA, 20 bp, Artificial Sequence
  Synthetic Construct
ctgacctgtcagctggccat

SEQ ID NO: 31 - DNA, 20 bp, Artificial Sequence
  Synthetic Construct
cagaacccacagttggccac

SEQ ID NO: 32 - DNA, 20 bp, Artificial Sequence
  Synthetic Construct
ctgatgtcgcagttgtccat

SEQ ID NO: 33 - DNA, 20 bp, Artificial Sequence
  Synthetic Construct
gtgaagtctcagttggacat

SEQ ID NO: 34 - DNA, 20 bp, Artificial Sequence
  Synthetic Construct
ctgagctggcagatggacat

SEQ ID NO: 35 - DNA, 20 bp, Artificial Sequence
  Synthetic Construct
ctgaactggaaggtggccag

SEQ ID NO: 36 - DNA, 20 bp, Artificial Sequence
  Synthetic Construct
ctgaacttggagctggccaa

SEQ ID NO: 37 - DNA, 20 bp, Artificial Sequence
  Synthetic Construct
ctgaagtcgaggttggccac

SEQ ID NO: 38 - DNA, 20 bp, Artificial Sequence
  Synthetic Construct
ctgacgtcccaggtggccat

SEQ ID NO: 39 - DNA, 20 bp, Artificial Sequence
  Synthetic Construct
ctgcaatcacagttggccct

SEQ ID NO: 40 - DNA, 20 bp, Artificial Sequence
  Synthetic Construct
cagaacatgcagttgtccat

SEQ ID NO: 41 - DNA, 20 bp, Artificial Sequence
  Synthetic Construct
ctcaactccctggtggccat

SEQ ID NO: 42 - DNA, 20 bp, Artificial Sequence
  Synthetic Construct
ctgaaatgtcagttggccct

SEQ ID NO: 43 - DNA, 20 bp, Artificial Sequence
  Synthetic Construct
ctgagctccctgctggccat

SEQ ID NO: 44 - DNA, 20 bp, Artificial Sequence
  Synthetic Construct
ttgagctcccagttgcccat

SEQ ID NO: 45 - DNA, 20 bp, Artificial Sequence
  Synthetic Construct
ctgaaatggctgatggccat

SEQ ID NO: 46 - DNA, 20 bp, Artificial Sequence
  Synthetic Construct
ctgaatgcacagttggccaa

SEQ ID NO: 47 - DNA, 20 bp, Artificial Sequence
  Synthetic Construct
ctgacctcccagatggccac

SEQ ID NO: 48 - DNA, 20 bp, Artificial Sequence
  Synthetic Construct
ctgaaatcctagttggccct

SEQ ID NO: 49 - DNA, 20 bp, Artificial Sequence
  Synthetic Construct
ctggactcccacatggccat

SEQ ID NO: 50 - DNA, 20 bp, Artificial Sequence
  Synthetic Construct
ctgatgtcgcacgtggccat

SEQ ID NO: 51 - DNA, 20 bp, Artificial Sequence
  Synthetic Construct
ctgaaacagcagttagccat

SEQ ID NO: 52 - DNA, 20 bp, Artificial Sequence
  Synthetic Construct
ctgaaacctcagttggtcat

SEQ ID NO: 53 - DNA, 20 bp, Artificial Sequence
  Synthetic Construct
ctgaactagtggttggcaat

SEQ ID NO: 54 - DNA, 20 bp, Artificial Sequence
  Synthetic Construct
cagaactagcagttggtaat

SEQ ID NO: 55 - DNA, 20 bp, Artificial Sequence
  Synthetic Construct
ctcagctcacagtgggccat

SEQ ID NO: 56 - DNA, 20 bp, Artificial Sequence
  Synthetic Construct
ctcaaatggcagtgggccat

SEQ ID NO: 57 - DNA, 20 bp, Artificial Sequence
  Synthetic Construct
ctgaactgtgagttgtccat

SEQ ID NO: 58 - DNA, 20 bp, Artificial Sequence
  Synthetic Construct
ctgaactggaacttggcgat

SEQ ID NO: 59 - DNA, 20 bp, Artificial Sequence
  Synthetic Construct
ctgaactcccagtgggccac

SEQ ID NO: 60 - DNA, 20 bp, Artificial Sequence
  Synthetic Construct
ctgaacttgaatttgcccat

(19) United States
(12) Patent Application Publication
(10) Pub. No.: US 2021/0228630 A1
(43) Pub. Date: Jul. 29, 2021

(54) GENERATION OF KNOCK-OUT PRIMARY AND EXPANDED HUMAN NK CELLS USING CAS9 RIBONUCLEOPROTEINS

(71) Applicant: RESEARCH INSTITUTE AT NATIONWIDE CHILDREN'S HOSPITAL, Columbus, OH (US)

(72) Inventors: Dean Anthony LEE, Canal Winchester, OH (US); Meisam NAEIMI KARAROUDI, Columbus, OH (US)

(21) Appl. No.: 17/055,837

(22) PCT Filed: May 16, 2019
(86) PCT No.: PCT/US2019/032670

§ 371 Date: Nov. 16, 2020

Related U.S. Application Data
(60) Provisional application No. 62/672,368, filed on May 16, 2018.

(51) Int. Cl. A61K 35/17; A61K 31/7088; C12N 9/10; C12N 9/22; C12N 5/0783

(52) U.S. Cl. CPC A61K 35/17; A61K 31/7088; C12N 2501/2312; C12N 9/22; C12N 5/0646; C12N 9/1048

(57) ABSTRACT

Disclosed are compositions and methods for genetically engineering NK cells.

### I. BACKGROUND

Cancer immunotherapy has been advanced in recent years. Genetically-modified chimeric antigen receptor (CAR) T cells are an excellent example of engineered immune cells successfully deployed in cancer immunotherapy. These cells were recently approved by the FDA for treatment against CD19+B cell malignancies, but success has so far been limited to diseases bearing a few targetable antigens, and targeting such limited antigenic repertoires is prone to failure by immune escape. Furthermore, CAR T cells have been focused on the use of autologous T cells because of the risk of graft-versus-host disease caused by allogeneic T cells. In contrast, NK cells are able to kill tumor targets in an antigen-independent manner and do not cause GvHD, which makes them a good candidate for cancer immunotherapy.

CRISPR/Cas9 technology has been used recently in engineering immune cells, but genetically reprogramming NK cells with plasmids has always been challenging. This has been due to difficulties in transgene delivery in a DNA dependent manner such as lentiviral and retroviral transduction causing substantial procedure-associated NK cell apoptosis and the limited production of genetically engineered NK cells. What are needed are new methods of genetically engineering NK cells.

### II. SUMMARY

Disclosed are methods and compositions related to genetically modified NK cells.

In one aspect, disclosed here are methods of genetically modifying an NK cell (such as, for example a primary or expanded NK cell) comprising obtaining guide RNA (gRNA) specific for a target DNA sequence in the NK cell (such as, for example, transforming growth factor-β receptor 2 (TGFBR2) or hypoxanthine phosphoribosyltransferase 1 (HPRT1); and b) introducing via electroporation into a target NK cell, a ribonucleoprotein (RNP) complex comprising a class 2 CRISPR/Cas endonuclease (Cas9) complexed with a corresponding CRISPR/Cas guide RNA that hybridizes to the target sequence within the genomic DNA of the NK cell.

Also disclosed herein are methods of any preceding aspect wherein the genome of the NK cell is modified by insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof.

In one aspect, disclosed herein are methods of genetically modifying an NK cell of any preceding aspect, wherein the NK cells (for example, primary or expanded NK cells) are incubated in the presence of IL-2 and/or irradiated feeder cells for 4, 5, 6, or 7 days prior to transduction (such as, electroporation).

Also disclosed herein are methods of genetically modifying an NK cell of any preceding aspect, further comprising expanding the modified NK cells with irradiated membrane bound interleukin-21 (mbIL-21) expressing feeder cells following electroporation.

In one aspect, disclosed herein are modified NK cells made be the method of any preceding aspect. In one aspect, the modified NK cell can comprise a knockout of the gene encoding the transforming growth factor-β receptor 2 (TGFBR2) or hypoxanthine phosphoribosyltransferase 1 (HPRT1).

Also disclosed herein are methods of treating a cancer comprising administering to a subject with a cancer the modified NK cell of any preceding aspect.

In one aspect, disclosed herein are methods of adoptively transferring an engineered NK cells to a subject in need thereof said method comprising a) obtaining a target NK cell (such as a primary NK cell or expanded NK cell) to be modified; b) obtaining gRNA specific for a target DNA sequence; c) introducing via electroporation into the target NK cell, a RNP complex comprising a class 2 CRISPR/Cas endonuclease (Cas9) complexed with a corresponding CRISPR/Cas gRNA that hybridizes to the target sequence within the genomic DNA of the target NK cell creating an engineered NK cell; and d) transferring the engineered NK cell into the subject.

Also disclosed herein are methods of adoptively transferring an engineered NK cells to a subject in need thereof wherein the NK cell is a primary NK cell (such as, for example, an autologous NK cell, or NK cell from an allogeneic donor source) that has been modified ex vivo and after modification transferred to the subject.

In one aspect, disclosed herein are methods of adoptively transferring an engineered NK cells to a subject in need thereof of any preceding aspect, wherein the NK cell is expanded with irradiated mbIL-21 expressing feeder cells or the administration of IL-21 prior to, concurrent with, or following administration of the modified NK cells to the subject.

In one aspect, disclosed herein are methods of adoptively transferring an engineered NK cells to a subject in need thereof, wherein the subject receiving the adoptively transferred modified NK cells has a cancer.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the electroporation efficiency of siRNA and plasmid DNA expressing GFP in NK cells using EN-138 program. As seen here, the NK cell viability is 77.5% and 35% of live cells were GFP positive.

FIG. 2 shows viability and efficiency of another one of the 16 programs (DN-100) tested for electroporation optimization.

FIG. 3 shows Cas9/RNPs-mediated TGFBR2 knockout in expended (a) Primary NK cells (b) measured by T7E1 Mutation Assay. T7E1 enzyme recognizes and cleaves mismatched DNA. Each small band (blue arrows) represents digested DNA fragments which carry an indel.

FIG. 4 shows Cas9/RNPs-mediated HPRT disruption in expanded NK cells measured by T7E1 Mutation assay.

FIG. 5 shows the mRNA expression level of TGFBR2 ectodomain in CRISPR modified NK cells introduced by Cas9/RNPs (gRNA1+gRNA2) using RT-PCR. GAPDH was used as an endogenous control gene. The reduction in RNA levels indicates a disruption of TGFBR2 gene.

FIG. 6A shows a cytotoxicity assay of Cas9/RNPs modified (gRNA1+gRNA2, gRNA2 and gRNA3) cells shows that overnight incubation of the cells with TGFB does not decrease significantly their ability to lyse DAOY cells.

FIG. 6B shows that when compared with non-modified NK cells, Cas9/RNP modified cells (gRNA2 and gRNA3) are less sensitive to TGFB.

FIG. 7 shows Exon 2 of the SOCS3 gene and gRNAs used to target Exon 2 of the SOCS3 gene.

FIG. 8 shows the relative Normalized expression level of Socs3 in knocked out NK cells compare to wild type NK.

FIGS. 9A, 9B, 9C, and 9D show the better expansion and cytotoxicity of SOCS3-KO NK cells. FIG. 9A shows Incucyte results of SOCS3-KO NK cells against AML. FIGS. 9B and 9C show cytotoxicity results from 3 donors against DAOY cells (9B) and the neuroblastoma cell line NB1643 (9C). FIG. 9D shows the actual number of dead cells for each cell line and treatment condition in FIGS. 9B and 9C.

FIG. 10 shows proliferation analysis showing the SOCS3 KO effect on NK cell expansion.

FIG. 11 shows CD38 expression on wild-type and CD38-knock out NK cells.

FIG. 12 shows resistance to daratumumab-mediated fratricide.

FIG. 13 shows that the Cas9/RNP platform successfully targets the AAVS1 locus in NK cells.

FIG. 14 shows the integration of mCherry reporter gene in to AAVS1 locus of human primary NK cells was evaluated using PCR.

FIG. 15 shows the stable gene expression of mCherry post expansion and sorting was studied using flow cytometry and florescent microscopy. *The results represent 2 out of 12 designed AAV constructs.

FIG. 16 shows the stable gene expression of mCherry post expansion and sorting of human primary NK cells using different culture conditions was evaluated using flow cytometry. Primary NK cells were electroporated with CAS9/RNP and transduced with 300K MOI of AAV6 SS800-mCherry and cultured in RPMI media+fetal bovine serum (FBS) or serum free AIMV media.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

A DNA sequence that “encodes” a particular RNA is a DNA nucleic acid sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is wild type (and naturally occurring).

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF GENETICALLY MODIFYING NK CELLS

Genetically reprogramming NK cells with plasmids has always been challenging due to difficulties in transgene delivery in a DNA dependent manner such as lentiviral and retroviral transduction causing substantial procedure-associated NK cell apoptosis and the limited production of genetically engineered NK cells. Described herein are methods for using a DNA-free genome editing of primary and expanded human NK cells utilizing endonuclease ribonucleoprotein complexes (such as, for example, Cas9/RNPs) to reprogram (i.e., engineer or modify) NK cells.

Endonuclease/RNPs (for example, a Cas9/RNP) are comprised of three components, recombinant endonuclease protein (for example, a Cas9 endonuclease) complexed with a CRISPR loci. The endonuclease complexed to the CRISPR loci can be referred to as a CRISPR/Cas guide RNA. The CRISPR loci comprises a synthetic single-guide RNA (gRNA) comprised of a RNA that can hybridize to a target sequence complexed complementary repeat RNA (crRNA) and trans complementary repeat RNA (tracrRNA). Accordingly the CRISPR/Cas guide RNA hybridizes to a target sequence within the genomic DNA of the cell. In some cases, the class 2 CRISPR/Cas endonuclease is a type II CRISPR/Cas endonuclease. In some cases, the class 2 CRISPR/Cas endonuclease is a Cas9 polypeptide and the corresponding CRISPR/Cas guide RNA is a Cas9 guide RNA. These Cas9/RNPs are capable of cleaving genomic targets with higher efficiency as compared to foreign DNA-dependent approaches due to their delivery as functional complexes. Additionally, rapid clearance of Cas9/RNPs from the cells can reduce the off-target effects such as induction of apoptosis. Accordingly, in one aspect, disclosed here are methods of genetically modifying an NK cell comprising obtaining guide RNA (gRNA) specific for a target DNA sequence in the NK cell; and b) transducing (for example, introducing via electroporation) into a target NK cell, a ribonucleoprotein (RNP) complex comprising a class 2 CRISPR/Cas endonuclease (Cas9) complexed with a corresponding CRISPR/Cas guide RNA that hybridizes to the target sequence within the genomic DNA of the NK cell.

It is understood and herein contemplated that to target the Cas9 nuclease activity to the target site and also cleave the donor plasmid to allow for recombination of the donor transgene into the host DNA, a crispr RNA (crRNA) is used. In some cases the crRNA is combined with a tracrRNA to form guide RNA (gRNA). The disclosed plasmids use AAV integration, intron 1 of the protein phosphatase 1, regulatory subunit 12C (PPP1R12C) gene on human chromosome 19, which is referred to the AAVS1, as the target site for the integration of the transgene. This locus is a “safe harbor gene” and allows stable, long-term transgene expression in many cell types. As disruption of PPP1R12C is not associated with any known disease, the AAVS1 locus is often considered a safe-harbor for transgene targeting. Because the AAVS1 site is being used as the target location, the CRSPR RNA (crRNA) must target said DNA. Herein, the guide RNA used in the disclosed plasmids comprises GGGGCCACTAGGGACAGGAT (SEQ ID NO: 9) or any 10 nucleotide sense or antisense contiguous fragment thereof. While AAVS1 is used for exemplary purposes here, it is understood and herein contemplated that other “safe harbor genes” can be used with equivalent results and can be substituted for AAVS1 if more appropriate given the particular cell type being transfected or the transgene. Examples of other safe harbor genes, include but are not limited to C—C chemokine receptor type 5 (CCR5), the ROSA26 locus, and TRAC.

It is understood and herein contemplated that there can be size limits on the donor transgene construct size delivered to the target genome. One method of increasing the allowable size of the transgene is to create additional room by exchanging the Cas9 of Streptococcus pyogenes (SpCas9) typically used for a synthetic Cas9, or Cas9 from a different bacterial source. Substitution of the Cas9 can also be used to increase the targeting specificity so less gRNA needs to be used. Thus, for example, the Cas9 can be derived from Staphylococcus aureus (SaCas9), Acidaminococcus sp. (AsCpf1), Lachnospiracase bacterium (LbCpf1), Neisseria meningitidis (NmCas9), Streptococcus thermophilus (StCas9), Campylobacter jejuni (CjCas9), enhanced SpCas9 (eSpCas9), SpCas9-HF1, Fokl-Fused dCas9, expanded Cas9 (xCas9), and/or catalytically dead Cas9 (dCas9).

It is understood and herein contemplated that the use of a particular Cas9 can change the PAM sequence which the Cas9 endonuclease (or alternative) uses to screen for targets. As used herein, suitable PAM sequences comprises NGG (SpCas9 PAM) NNGRRT (SaCas9 PAM) NNNNGATT (NmCAs9 PAM), NNNNRYAC (CjCas9 PAM), NNAGAAW (St), TTTV (LbCpf1 PAM and AsCpf1 PAM); TYCV (LbCpf1 PAM variant and AsCpf1 PAM variant); where N can be any nucleotide; V=A, C, or G; Y=C or T; W=A or T; and R=A or G.

To make the RNP complex, crRNA and tracrRNA can be mixed at a 1:1, 2:1, or 1:2 ratio of concentrations between about 50 μM and about 500 μM (for example, 50, 60, 70, 80, 90, 100, 125, 150, 175, 200, 225, 250, 275, 300, 325, 35, 375, 400, 425, 450, 475, or 500 μM), preferably between 100 μM and about 300 μM, most preferably about 200 μM at 95 C for about 5 min to form a crRNA:tracrRNA complex (i.e., the guide RNA). The crRNA:tracrRNA complex can then be mixed with between about 20 μM and about 50 μM (for example 21, 22, 23, 24, 25, 26, 27, 28, 29, 30, 31, 32, 33, 34, 35, 36, 37, 38, 39, 40, 41, 42, 43, 44, 45, 46, 47 48, 49, or 50 μM) final dilution of a Cas endonuclease (such as, for example, Cas9).

Once bound to the target sequence in the target cell, the CRISPR loci can modify the genome by introducing into the target DNA insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof. Thus, the disclosed methods can be used to generate knock-outs or knock-ins when combined with DNA for homologous recombination. It is shown herein that transduction via electroporation of Cas9/RNPs is an easy and relatively efficient method that overcomes the previous constraints of genetic modification in NK cells.

It is understood and herein contemplated that the disclosed methods can be utilized with any cell type including natural killer cells (NK cells), T cells, B cells, macrophages, fibroblasts, osteoblasts, hepatocytes, neuronal cells, epithelial cells, and/or muscle cells. Human NK cells are a subset of peripheral blood lymphocytes defined by the expression of CD56 or CD16 and the absence of T cell receptor (CD3). NK cells sense and kill target cells that lack major histocompatibility complex (MHC)-class I molecules. NK cell activating receptors include, among others, the natural cytotoxicity receptors (NKp30, NKp44 and NKp46), and lectin-like receptors NKG2D and DNAM-1. Their ligands are expressed on stressed, transformed, or infected cells but not on normal cells, making normal cells resistant to NK cell killing. NK cell activation is negatively regulated via inhibitory receptors, such as killer immunoglobin (Ig)-like receptors (KIRs), NKG2A/CD94, TGFβ, and leukocyte Ig-like receptor-1 (LIR-1). In one aspect, the target cells can be primary NK cells from a donor source (such as, for example, an allogeneic donor source for an adoptive transfer therapy or an autologous donor source (i.e., the ultimate recipient of the modified NK cells), NK cell line (including, but not limited to NK RPMI8866; HFWT, K562, and EBV-LCL), or from a source of expanded NK cells derived a primary NK cell source or NK cell line.

Prior to the transduction of the NK cells, the NK cell can be incubated in a media suitable for the propagation of NK cells. It is understood and herein contemplated that the culturing conditions can comprise the addition of cytokines, antibodies, and/or feeder cells. Thus, in one aspect, disclosed herein are methods of genetically modifying an NK cell, further comprising incubating the NK cells for 1, 2, 3, 4, 5, 6, 7, 8 9, 10, 11, 12, 13, or 14 days prior to transducing the cells in media that supports the propagation of NK cells; wherein the media further comprises cytokines, antibodies, and/or feeder cells. For example, the media can comprise IL-2, IL-12, IL-15, IL-18, and/or IL-21. In one aspect, the media can also comprise anti-CD3 antibody. In one aspect, the feeder cells can be purified from feeder cells that stimulate NK cells. NK cell stimulating feeder cells for use in the claimed invention, disclosed herein can be either irradiated autologous or allogeneic peripheral blood mononuclear cells (PBMCs) or nonirradiated autologous or PBMCs; RPMI8866; HFWT, K562; K562 cells transfected with membrane bound IL-15, and 41BBL, or IL-21 or any combination thereof; or EBV-LCL. In some aspects, the NK cell feeder cells provided in combination with a solution of IL-21, IL-15, and/or 41BBL. Feeder cells can be seeded in the culture of NK cells at a 1:2, 1:1, or 2:1 ratio. The It is understood and herein contemplated that the period of culturing can be between 1 and 14 days post electroporation (ie, 1, 2, 3, 4, 5, 6, 7, 8, 9, 10, 11, 12, 13, or 14 days), preferably between 3 and 7 days, most preferably between 4 and 6 days.

It is understood and herein contemplated that the incubation conditions for primary NK cells and expanded NK cells can be different. In one aspect, the culturing of primary NK cells prior to electroporation comprises media and cytokines (such as, for example, IL-2, IL-12, IL-15, IL-18, and/or IL-21) and/or anti-CD3 antibody for less than 5 days (for example 1, 2, 3, or 4 days). For expanded NK cells the culturing can occur in the presence of NK feeder cells (at for example, a 1:1 ratio) in addition to or in lieu of cytokines (such as, for example, IL-2, IL-12, IL-15, IL-18, and/or IL-21) and/or anti-CD3 antibody. Culturing of expanded NK cells can occur for 1, 2, 3, 4, 5, 6, 7, 8, 9, or 10 days prior to transduction. Thus, in one aspect, disclosed herein are methods of genetically modifying an NK cell comprising incubating primary NK cells for 4 days in the presence of IL-2 prior to electroporation or incubating expanded NK cells in the presence of irradiated feeder cells for 6, 7, 8, 9, 10, 11, 12, 13, 14, 15, 16, 17, 18, 19, 20, 21, 22, 23, 24, 30, 36, 42, 48, 54, 60 hours, 3, 4, 5, 6, or 7 days prior to electroporation.

It is understood and herein contemplated that methods of transduction to modify NK cells in the disclosed methods is limited. Due to their immune function, NK cells are resistant to viral and bacterial vectors and the induction of NK cell apoptosis by said vectors. Thus, prior to the present methods CRISPR/Cas modification of NK cells has been unsuccessful. To circumvent problems with viral vectors, the disclosed methods transform the target NK cells using electroporation. Electroporation is a technique in which an electric field is applied to cells to increase the permeability of the cell membrane. The application of the electric filed cause a charge gradient across the membrane which draws the charged molecules such as, nucleic acid, across the cell membrane. Thus, in one aspect, disclosed herein are methods of genetically modifying an NK cell comprising obtaining guide RNA (gRNA) specific for a target DNA sequence in the NK cell; and b) introducing via electroporation into a target NK cell, a ribonucleoprotein (RNP) complex comprising a class 2 CRISPR/Cas endonuclease (Cas9) complexed with a corresponding CRISPR/Cas guide RNA that hybridizes to the target sequence within the genomic DNA of the NK cell.

Following transduction (e.g., electroporation) of the NK cell, the now modified NK cell can be propagated in a media comprising feeder cells that stimulate the modified NK cells. Thus, the modified cells retain viability and proliferative potential, as they are able to be expanded post-electroporation using irradiated feeder cells. NK cell stimulating feeder cells for use in the claimed invention, disclosed herein can be either irradiated autologous or allogeneic peripheral blood mononuclear cells (PBMCs) or nonirradiated autologous or PBMCs; RPMI8866; HFWT, K562; K562 cells transfected with membrane bound IL-15, and 41BBL, or IL-21 or any combination thereof; or EBV-LCL. In some aspects, the NK cell feeder cells provided in combination with a solution of IL-21, IL-15, and/or 41BBL. Feeder cells can be seeded in the culture of NK cells at a 1:2, 1:1, or 2:1 ratio. The It is understood and herein contemplated that the period of culturing can be between 1 and 14 days post electroporation (i.e, 1, 2, 3, 4, 5, 6, 7, 8, 9, 10, 11, 12, 13, or 14 days), preferably between 3 and 7 days, most preferably between 4 and 6 days. In some aspect, the media for culturing the modified NK cells can further comprise cytokines such as, for example, IL-2, IL-12, IL-15, IL-18, and/or IL-21.

In one aspect, it is understood and herein contemplated that one goal of the disclosed methods of genetically modifying an NK cell is to produce a modified NK cell. Accordingly, disclosed herein are modified NK cells made by the disclosed methods.

As noted above, NK cell activation is negatively regulated via inhibitory receptors, such as killer immunoglobin (Ig)-like receptors (KIRs), NKG2A/CD94, TGFβ, and leukocyte Ig-like receptor-1 (LIR-1). Engagement of one inhibitory receptor may be sufficient to prevent target lysis. Hence NK cells efficiently target cells that express many stress-induced ligands, and few MHC class I ligands. TGFβ is a major immunosuppressive cytokine which inhibits the activation and functions of NK cells. Thus, it is understood and herein contemplated that one modification of NK cells that would be advantageous is the suppression of inhibitory receptors, such as killer immunoglobin (Ig)-like receptors (KIRs), NKG2A/CD94, TGFβ, and leukocyte Ig-like receptor-1 (LIR-1) so the negative regulation of NK cells would be suppressed. Such modified cells would be very useful in immunotherapy of any disease or condition that could be treated with the addition of NK cells. Thus, in one aspect, disclosed herein are genetically modified NK cell comprising a knockout of the gene encoding the transforming growth factor-β receptor 2 (TGFBR2) or hypoxanthine phosphoribosyltransferase 1 (HPRT1).

As noted throughout the present disclosure, the disclosed modified NK cells are ideally suited for use in immunotherapy such as the adoptive transfer of modified (i.e, engineered NK cells to a subject in need thereof. Thus, in one aspect, disclosed herein are methods of adoptively transferring an engineered NK cells to a subject in need thereof said method comprising a) obtaining a target NK cell to be modified; b) obtaining gRNA specific for a target DNA sequence; c) introducing via electroporation into the target NK cell, a RNP complex comprising a class 2 CRISPR/Cas endonuclease (Cas9) complexed with a corresponding CRISPR/Cas gRNA that hybridizes to the target sequence within the genomic DNA of the target NK cell creating an engineered NK cell; and d) transferring the engineered NK cell into the subject.

In one aspect, the modified NK cells used in the disclosed immunotherapy methods can be primary NK cells from a donor source (such as, for example, an allogeneic donor source for an adoptive transfer therapy or an autologous donor source (i.e., the ultimate recipient of the modified NK cells), NK cell line (including, but not limited to NK RPMI8866; HFWT, K562, and EBV-LCL), or from a source of expanded NK cells derived a primary NK cell source or NK cell line. Because primary NK cells can be used, it is understood and herein contemplated that the disclosed modifications of the NK cell can occur ex vivo or in vitro.

Following transduction of the NK cells, the modified NK cells can be expanded and stimulated prior to administration of the modified (i.e., engineered) NK cells to the subject. For example, disclosed herein are methods of adoptively transferring NK cells to a subject in need thereof wherein the NK cell is expanded with irradiated mbIL-21 expressing feeder cells prior to administration to the subject. In some aspect, it is understood and herein contemplated that eh stimulation and expansion of the modified (i.e, engineered) NK cells can occur in vivo following or concurrent with the administration of the modified NK cells to the subject. Accordingly disclosed herein are immunotherapy methods wherein the NK cells are expanded in the subject following transfer of the NK cells to the subject via the administration of IL-21 or irradiated mbIL-21 expressing feeder cells.

It has been indicated that targeting the TGFβ pathway can increase immune cell functions. The region encoding TGBR2 ectodomain which binds TGFβ was targeted. The representative results show a significant decrease in the level of mRNA expression of this gene and further demonstrate that the modified NK cells become resistant to TGFβ. Accordingly, disclosed herein are immunotherapy methods wherein the RNP complex targets the TGFRB2 or HPRT1 gene.

It is understood and herein contemplated that the disclosed modified NK cell and adoptive transfer methods of the modified NK cells can be effective immunotherapy against a cancer. The disclosed methods and compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. Accordingly, disclosed herein, in one aspect, are methods of treating a cancer in a subject comprising administering to the subject an NK cell that has been modified to comprise a knockout of the TGFBR2 gene.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

### 1. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 2. Nucleic Acids

There are a variety of molecules disclosed herein that are nucleic acid based, including for example the nucleic acids that encode, for example TGFβR2, or any of the nucleic acids disclosed herein for making TGFRβ2 knockouts, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the signaling pathways disclosed herein, for example TGFβR2, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Primers and Probes

Disclosed are compositions including primers and probes, which are capable of interacting with the disclosed nucleic acids, such as the TGFβR2 and/or HPRT1 as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the TGFβR2 and HPRT1 gene typically will be used to produce an amplified DNA product that contains a region of the TGFβR2 and HPRT1 gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

### 3. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osbome, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. coli lacZ gene, which encodes ß-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 4. Peptides

a) Protein Variants

Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) Methods

(1) Human NK Cell Purification and Expansion

Healthy donor buffy coats were obtained as source material from the Central Ohio Region American Red Cross. This research was determined to be exempt research by the Institutional Review Board of Nationwide Children's Hospital.

Isolate PBMCs from Buffy Coat. Briefly, Layer 35 mL of buffy coat sample on 15 ml Ficol-Paque. Centrifuge at 400×g for 20 minutes without brake. Wash the recovered PBMCs three times with PBS. NK cell can be isolated at this stage by RosettesSep.

Expand NK cells by stimulating with irradiated 10×10⁶ mbIL21-expressing feeder cells at Days 0, 7, and 14. Replace media with fresh AIMV or RPMI containing 10% FBS, 1% Glutamine, 1% Penicillin Streptomycin and 100 IU/mL of IL-2 for the entire media volume every other day.

(2) gRNA Design and Selection

Choose the specific genomic loci to target, using online tools e.g. NCBI, Ensemble. By way of example, using transforming growth factor beta receptor 2 (TGFBRR2) ectodomain. View record: PF08917; View InterPro: IPR015013; Position: 49-157 aa. Targeted Sequence: Exon 4 of TGFBR2 gene (ENSG00000163513)

To design your gRNAs, Use CRISPR design web tools such as http://crispr.mit.edu and ‘Benchling.com’. Enter in your DNA sequence chosen in step 2.1. Choose human (hg 19) as a target genome. CRISPR guides (20 nucleotides followed by a PAM sequence: NGG) are scanned from the sequence entered earlier. It also shows possible off-target matches throughout the selected genome. Choose the best three gRNAs which have the highest score, based on their on-target and off-target rates.

Table 1, shows the designed CRISPR RNAs to target exon 4 of TGFBR2 gene indicated by CRISPR design web tools.

TABLE 1
Three designed gRNAs to target exon 4 of TGFBR2 ectodomain as synthetic crRNA.

| gRNA NO. | gRNA sequence | Ordered as synthetic crRNA |
|---|---|---|
| gRNA1 | 5 CCCCTACCATGACTTTATTC 3 | /AltR1/rArGrUrCrArUrGrGrUrArGrG |
| | (SEQ ID NO: 1) | rGrGrArGrCrUrUrGrGrUrUrUrUrArG |
| | | rArGrCrUrArUrGrCrU/AltR2/ (SEQ |
| | | ID NO: 4) |
| gRNA2 | 5 ATTGCACTCATCAGAGCTAC 3 | /AltR1/rArUrUrGrCrArCrUrCrArUrCr |
| | (SEQ ID NO: 2) | ArGrArGrCrUrArCrGrUrUrUrUrArGr |
| | | ArGrCrUrArUrGrCrU/AltR2/ (SEQ ID |
| | | NO: 5) |
| gRNA3 | 5 AGTCATGGTAGGGGAGCTTG 3 | /AltR1/rArG rUrCrA rUrGrG |
| | (SEQ ID NO: 3) | rUrArGrGrGrG rArGrC rUrUrG |
| | | rGrUrUrUrUrA rGrArG rCrUrA |
| | | rUrGrCrU/AltR2/ (SEQ ID NO: 6) |

Order the CRISPR RNAs as synthetic sequence-specific crRNAs and order a conserved, transactivating RNA (tracrRNA) to interact through partial homology with your crRNA.

(3) Design Deletion Screening Primers

Design primers spanning the gRNA cleavage sites for T7E1 mutation assay. Use primers at least 100 bp away from the predicted cleavage site to ensure small insertion-deletion (indels) at the sgRNA target site appear on 1.5% agarose gel following mutation assay. Table 2. shows the primers which are used to amplify the TGFBR2 ectodomain.

TABLE 2
Primers used to amplify the TGFBR2 ectodomain gene

| | |
|---|---|
| TGFBR 2 | 5 GTC TGC TCC AGG TGA TGT TTA T3 |
| ectodomain | (SEQ ID NO: 7) |
| Primers FWD | |
| TGFBR2 | 5 GGG CCT GAG AAT CTG CAT TTA 3 |
| ectodomain | (SEQ ID NO: 8) |
| Primer REV | |

(4) Transduction of Human Primary and Expanded NK Cells

Transduction of Cas9/RNPs elements into NK is done by electroporation using 4D-Nucleofector System as follows:

(5) Cell Preparation

For primary NK cells, incubate freshly isolated NK cells in RPMI or AIMV medium in the presence of 100 IU/mL of IL-2 for 4 days and perform the electroporation at Day 5 (Replace the media every other day as described earlier and the day before transduction). This can be modified for expanded NK cells. For expanded NK cells, Stimulate the cells at day 0 with irradiated feeder cells at a ratio of 1:1 and perform the electroporation at Day 5 or 6 or 7. (Replace the media every other day as described earlier and the day before transduction). At the day of electroporation prepare a T25 flask filled with 8 ml fresh RPMI containing 100 IU/mL of IL-2 for cells undergoing electroporation and pre-incubate flasks in a humidified 37° C./5% CO2 incubator. Thawed cells or cells that have undergone 2^{nd} or 3^{rd} stimulation can be electroporated at any time after their recovery as described. Take 3-4×10⁶ cells per condition for 26 μL transduction mix as a very high concentration of NK cells in Nucleofector Solution enhances the transduction rate. Cells 3 times can be washed with PBS to remove all FBS, which commonly contains RNase activity. Spin them down each time at 300 g for 8 minutes. Consider 7 electroporation conditions for Cas/RNPs as single gRNA (gRNA1, gRNA2, gRNA3) and a combination of two gRNAs (gRNA1+gRNA2, gRNA1+gRNA3, gRNA2+gRNA3) and one control with no Cas9/RNPs.

(6) Form the crRNA:tracerRNA/Complex

Resuspend crRNAs (gRNA1, gRNA2 and gRNA3) and TracerRNA in 1×TE solution to final concentrations of 200 μM. Mix 2.2 μl of each 200 μM gRNA with 200 μM TracerRNA as shown in table 3. Heat the samples at 95° C. for 5 min and allow to cool on the bench top to room temperature (15-25° C.). Store resuspended RNAs and crRNA:tracerRNA/complex at −20° C. for later use.

TABLE 3
Form the crRNA:tracerRNA/complex using 200 μM RNAs

| Component | Amount (uL) |
|---|---|
| 200 μM crRNA | 2.2 |
| 200 μM Tracer RNA | 2.2 |
| IDTE Buffer | 5.6 |
| Final product | 10 |

(7) Form the RNP Complex

To save time, form the RNP complex during the washing step. For single crRNA:tracrRNA duplex reaction, dilute Cas9 Endonuclease to 36 μM as mentioned in Table 4.

TABLE 4
For single crRNA:tracrRNA duplex reaction,
Dilute Cas9 endonuclease to 36 μM.

| Component | Amount (μL) |
|---|---|
| PBS | 1 |
| crRNA:tracrRNA duplex (from step 4.2) | 2 (200 pmol) |
| Alt-R Cas9 endonuclease (61 μM stock) | 2 |
| Total volume | 5 ul |

For combination transduction of crRNA:tracrRNA duplexes dilute Cas9 Endonuclease to 36 μM as shown in Table 5.

TABLE 5
For combination transduction of crRNA:tracrRNA
duplexes dilute Cas9 endonuclease to 36 μM.

| Component) | Amount (μL) |
|---|---|
| PBS | 1 |
| crRNA:tracrRNA duplex (ex. gRNA1) | 1 (100 pmol) |
| crRNA:tracrRNA duplex (ex. gRNA2) | 1 (100 pmol) |
| Alt-R Cas9 endonuclease | 2 |
| Total volume | 5 μL |

Add Cas9 Endonuclease to crRNA:tracrRNA duplexes slowly while swirling pipette tip, over 30 s to 1 minute. Incubate the mixture at room temperature for 15-20 min. If you are not ready to use the mixture after incubation, keep the mixture on ice until use.

(8) Electroporation

Add entire supplement to the Nucleofector Solution P3 and keep it at room temperature. Resuspend the cell pellet (3-4×10⁶ cells) in 20 μl of P3 Primary 4D Nucleofector Solution. Avoid air bubbles while pipetting. The cells should not be left for a long time in P3 solution. Immediately add 5 μL of RNP complex to the cell suspension. Add 1 μl of 100 μM Cas9 electroporation Enhancer to the Cas9/RNPs/cell mix. Transfer Cas9/RNPs/cell mix into 20 μl Nucleocuvette Strips. Gently tap the Nucleocuvette Strips to make sure the sample covers the bottom of the strips. Start 4D-Nucleofector System and choose the EN-138 program.

(9) Post Transduction

Let the cells rest for 3 minutes in strips. Add 80 μL of the pre-equilibrated culture media to the cuvette and gently transfer the sample into flasks. 48 hours after transduction, extract genomic DNA from 5×10⁵ cells for the gene deletions screening. Amplify your gene of interest using the primers designed in step 3.2 with Taq DNA polymerase kits. Form PCR amplicon heteroduplexes for T7EI digestion and incubate the product for 30-60 minutes with a T7EI enzyme in 37° C. The T7EI assay is preferred for screening as it is fast, simple and provides clean electrophoresis results compared to using Surveyor assay. However, this method cannot detect insertions and deletions of ≤2 bases that are generated by non-homologous end joining (NHEJ) activity in Cas9 RNPs experiments.

Run the digested DNA on 1.5% agarose gel at 110 V for 30-45 minutes, every 15 minutes visualize your gel. Stimulate the rest of the cells with the mbIL21-expressing feeder cells at a ratio of 1:1. Five days after stimulation extract the RNA for gene expression level using qPCR.

Conduct Calcein assays as previously reported. Briefly, load target cells with calcein AM (in the example shown, 3 μg/mL/1,000,000 DAOY cells was used). Prepare NK cells for cytotoxicity assays by resting overnight in IL2 (100 IU/mL) plus or minus 10 ng/mL soluble TGFβ. Conduct Calcein assays in the same cytokines as the NK cells were rested in overnight.

b) Results:

(1) Electroporation EFFICIENCY:

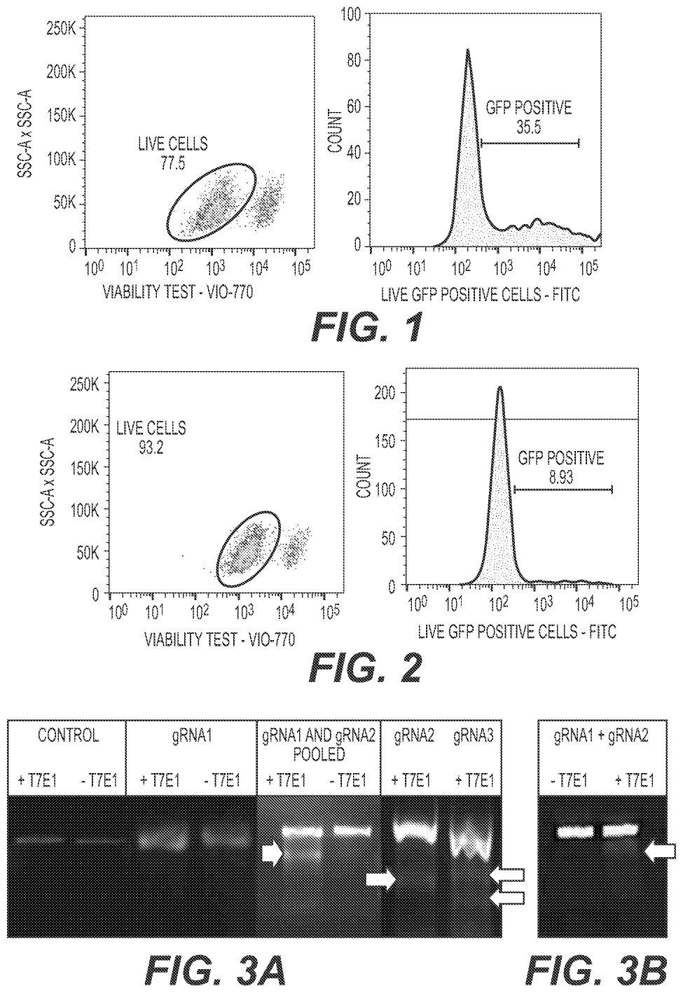

To optimize 4D-Nucleofection of Cas9/RNPs electroporation, 16 different programs were tested with transduction of GFP non-targeting siRNA and DNA plasmid into NK cells. Flow cytometry assay showed that the EN-138 had the highest percentage of cell viability and transduction efficiency (35% Live GFP positive cells) for both particles. (FIG. 1 & FIG. 2). Interestingly, the efficiency of using this program for Cas9/RNPs electroporation was higher as a 60% reduction in TGFBR2 mRNA expression level (FIG. 5) was observed.

(2) Mutation Assay

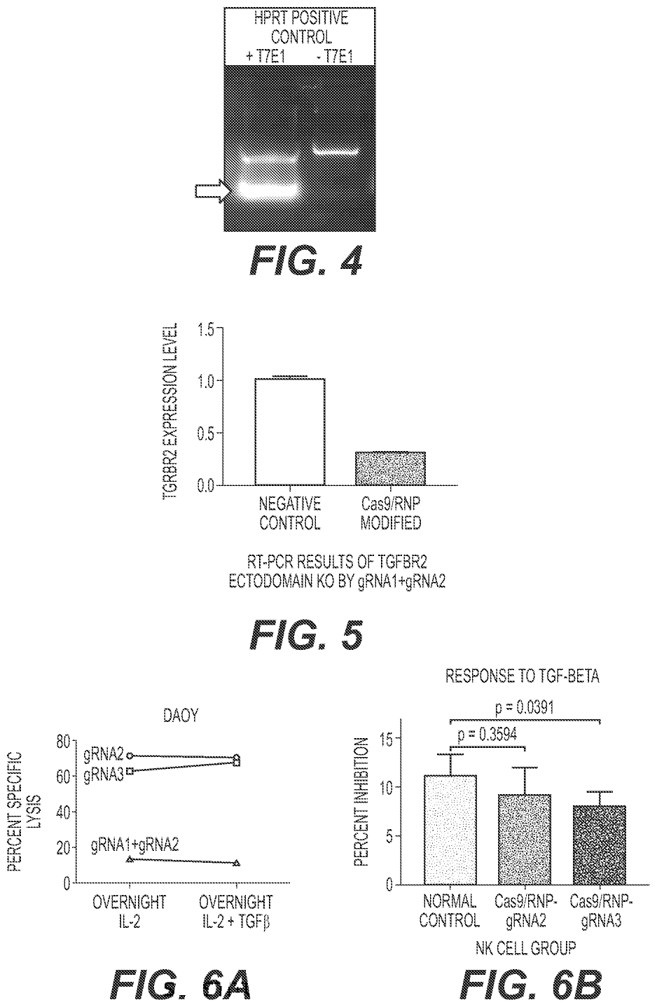

Cas9/RNPs containing gRNA2, gRNA1+gRNA2 and gRNA3 had successful TGFBR2 ectodomain gene knockout, but gRNA1 alone did not make any T7E1 detectable indels (FIG. 3). Additionally, FIG. 4 indicates successfully knockout of Human HPRT1 (hypoxanthine phosphoribosyltransferase 1) in expanded human NK cells using commercially provided gRNAs.

(3) Gene Expression Level Assay

As representative of the result, FIG. 5 shows the effect of Cas9/RNPs (gRNA1+gRNA2) on mRNA production level of TGFBR2 ectodomain, analyzed by RT-PCR. As seen in the graph, the mRNA expression level of the targeted gene significantly decreased.

(4) Cytotoxicity

As seen in FIG. 6, after incubating gRNA1+gRNA2, gRNA2 and gRNA3 Cas9/RNPs modified cells with TGFB, co-cultured with DAOY cells, the modified cells did not show any significant decrease in their cytotoxicity level in comparison to the control group which had IL-2 in the media overnight. This result demonstrates that the Cas9/RNPs modified cells retain their cytotoxicity function in presence of TGFB and shows that the modified cells became TGFB resistant.

(5) RNAseq Analysis

The RNAseq analysis in the naïve and expanded NK cells highlighted active DNA repair and replication machinery in IL-21 expanded NK cells. This indicates that the expended NK cells can be more open to genetic manipulation using Cas9/RNPs system.

c) Discussion:

Cas9-mediated genome engineering has revolutionized experimental and clinical medicine. Using this technique in T-cells has been successful, but DNA-dependent modification of NK cells has been challenging. In the CRISPR/Cas9 system, the DNA vector which carries the coding sequence for sgRNAs is put under the control of U6 or H1 promoters, since the resulting transcriptional process is either necessary nor desirable. DNA dependent transgene delivery such as lentiviral and retroviral transfection is poor due to substantial procedure-associated NK cell apoptosis, which limits efficient production of genetically engineered NK cells.

Therefore a synthetically preformed ribonucleoprotein (RNPs) complex and Cas9 protein were introduced as purified protein into primary and expanded NK cells.

This method allowed the elimination of capping, tailing and other transcriptional and translational processes started by RNA polymerase II which can cause substantial procedure-associated NK cell apoptosis as is considered to occur in DNA-dependent transduction methods. In addition, the method reported here uses purified Cas9 protein, increasing the on-target effects and decreasing the off-target impacts as Cas9/RNPs are active immediately following electroporation and are degraded quickly as well, providing an improvement to current protocols.

In summary, Cas9/RNPs can be used to genetically modify human primary and expanded NK cells for cancer immunotherapy utilizing the above described method. The results also demonstrated that a successful knockout of the TGFBR2 ectodomain gene leads to these modified NK cells becoming TGFB resistant.

Combining RNP delivery with a source of template DNA (such as naturally recombinogenic adeno-associated virus (AAV) donor vectors) can enable site-specific gene insertion by homologous recombination.

### 2. Example 2: Suppressor of Cytokine Signaling 3 (SOCS3)

Genetic modification of NK cells to enhance cancer immunotherapy has application to treat a wide range of cancers. Recently, a new strategy was developed in which CRISPR/Cas9 elements are introduced into NK cells as ribonucleoproteins (RNPs) via electroporation, followed by expansion on feeder cells expressing 4-1BBL and membrane-bound IL-21 to generate large numbers of genetically modified NK cells. This method was used to genetically modify several genes in primary and expanded NK cells including suppressor of cytokine signaling 3 (SOCS3). SOCS3 negatively regulates cytokine signaling through the JAK/STAT pathway. It was hypothesized that disruption of SOCS3 in primary NK cells using Cas9/RNPs could maintain STAT3 signaling levels and subsequently increase their proliferation and cytotoxic function.

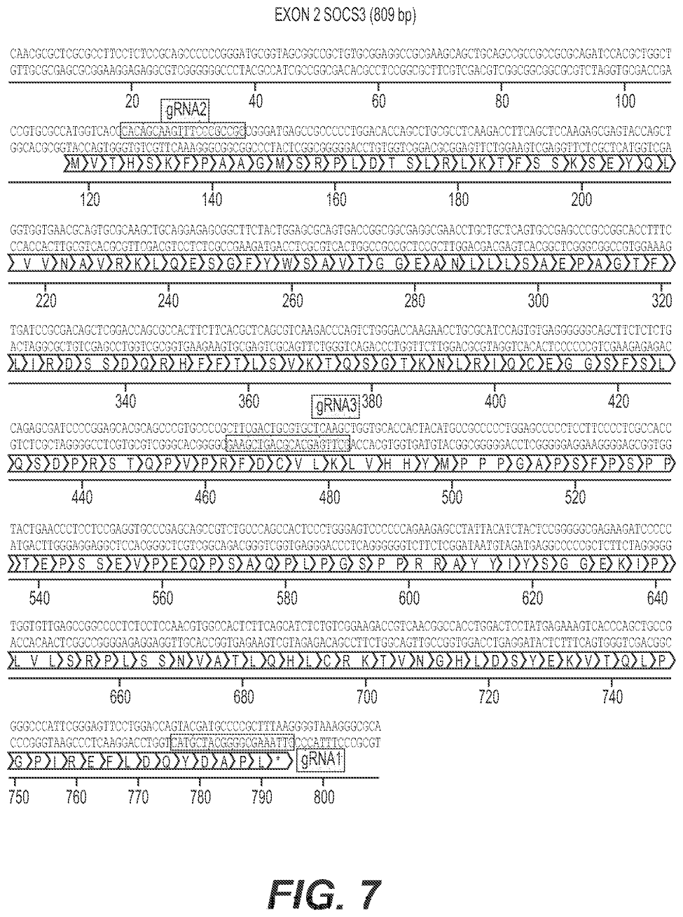

gRNAs were designed to target exon 2 (FIG. 7) of the SOCS3 gene and electroporated them along with Cas9 protein as Cas9/RNPs into primary NK cells using the Lonza 4D electroporator. Six different conditions of gRNAs were tested alone or in combination. The NK cells in the control group were electroporated with no Cas9/RNPs. After electroporation, the cells were rested in culture media supplemented with 100 IU of human IL-2 for 48 hours and then expanded using irradiated feeder cells. At day 7, an equal number of cells were restimulated with irradiated feeder cells to test the effect of SOCS3 KO on proliferation. Western blot was used to assay the knock out efficacy at the protein level. Calcein assay and IncuCyte Zoom (Essen) were performed to measure cytotoxicity against two cancer cell lines, K562 and Daoy.

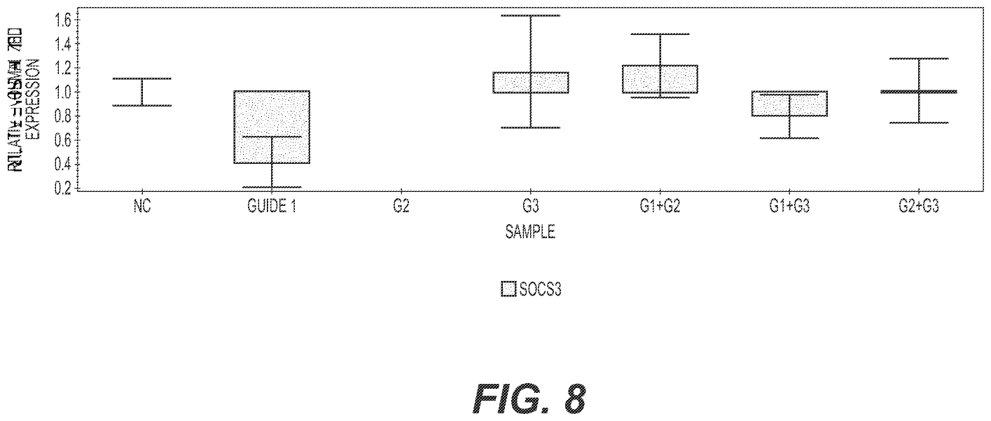

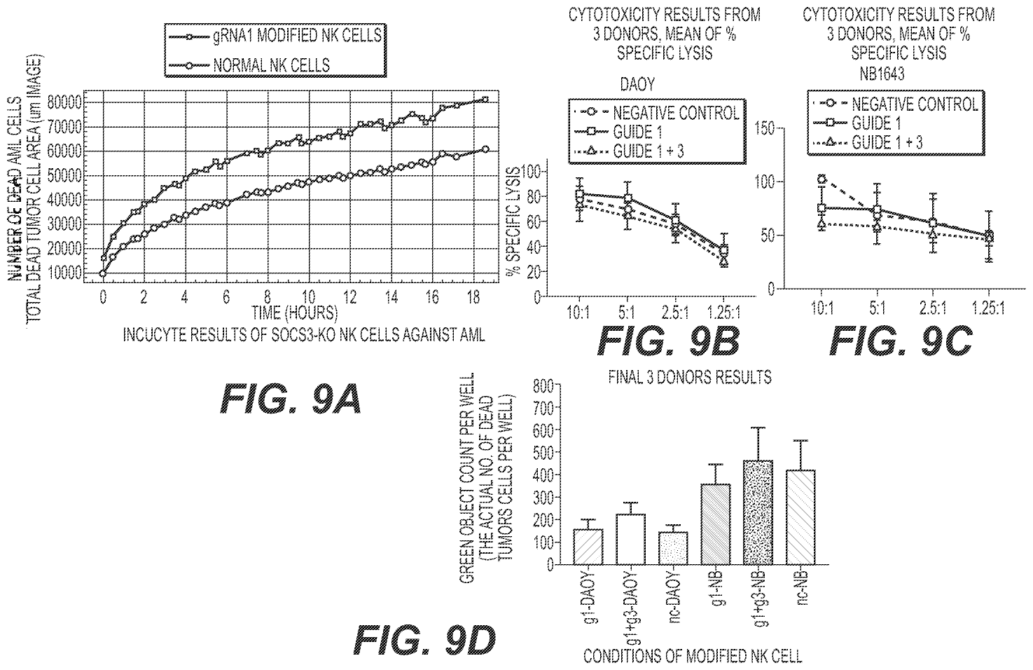

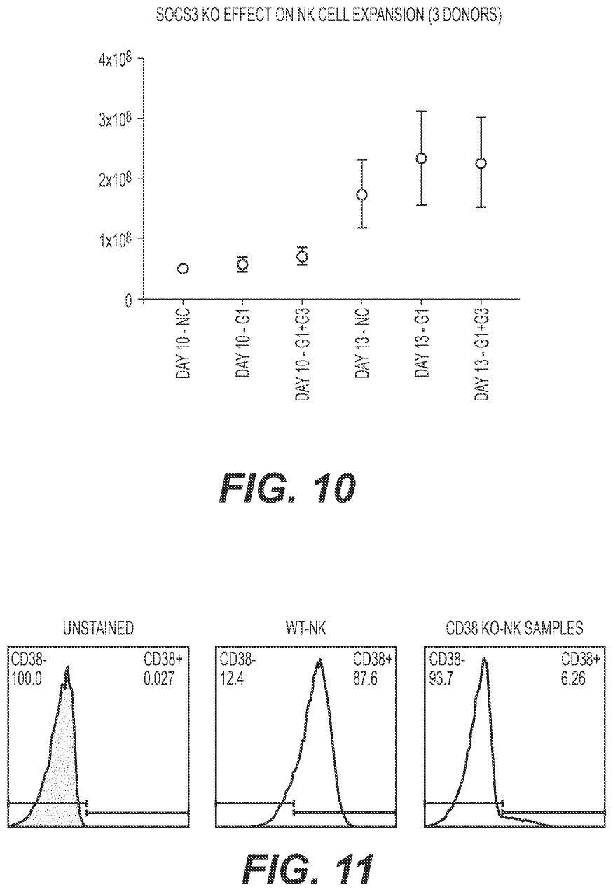

The results showed a significant reduction in SOCS3 protein levels in 3 conditions (gRNA1, gRNA3 and gRNA1+gRNA3) in comparison to the control group. The relative normalized expression of SOCS3 is shown in FIG. 8. The calcein assay and IncuCyte zoom showed the modified SOCS3 KO NK cells can kill the tumors more efficiently in comparison to the control in AML and Daoy cancer cell lines (FIGS. 9A, 9B, 9C, and 9D). gRNA 1 (G1) showed twice the killing of negative controls (NC). In particular, NC shows 80% killing at 10:1 whereas G1 shows 80% killing at 5:1 (FIGS. 9B and 9D). Neuroblastoma cells were not killed at a faster rate in SOCS3 knockouts (FIGS. 9C and 9D). Proliferation data showed the SOCS3 KO cells can grow faster than the control group (FIG. 10).

In conclusion, the data demonstrate the role of SOCS3 and JAK/STAT pathway in NK cell function and indicate that SOCS3 is a good target for genetic modification to improve cancer immunotherapy using NK cells.

### 3. Example 3: Generating CD38-KO NK Cells to Overcome Fratricide and Enhance ADCC

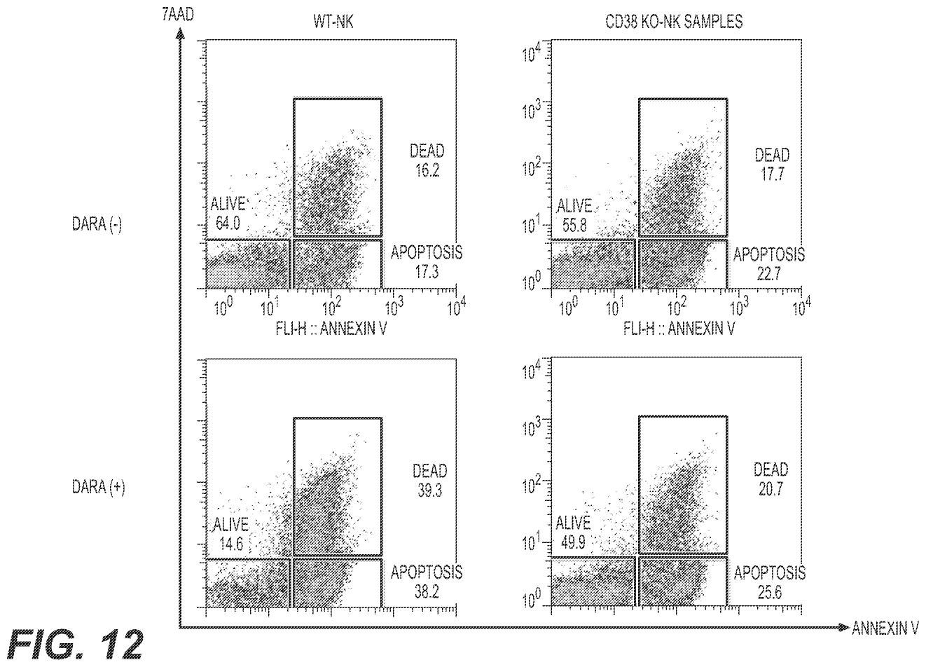

Natural killer cells play an important role in targeting CD38-expressing Multiple myeloma (MM) by anti-CD38 monoclonal antibody, daratumamb (DARA). To overcome fratricide of NK cells in DARA therapy, knock-out NK cells using Cas9/RNP were generated. Combination therapy of ex vivo-expanded autologous knock-out NK cells with DARA showed a significant improvement in DARA induced tumor cell killing (FIGS. 11 and 12).

### 4. Example 4: AAVS1

This methodology was used to target AAVS1 gene as a safe harbor to be used as an integration site for any genes of interest including CARs and reporter genes into the genome of the primary NK cells.

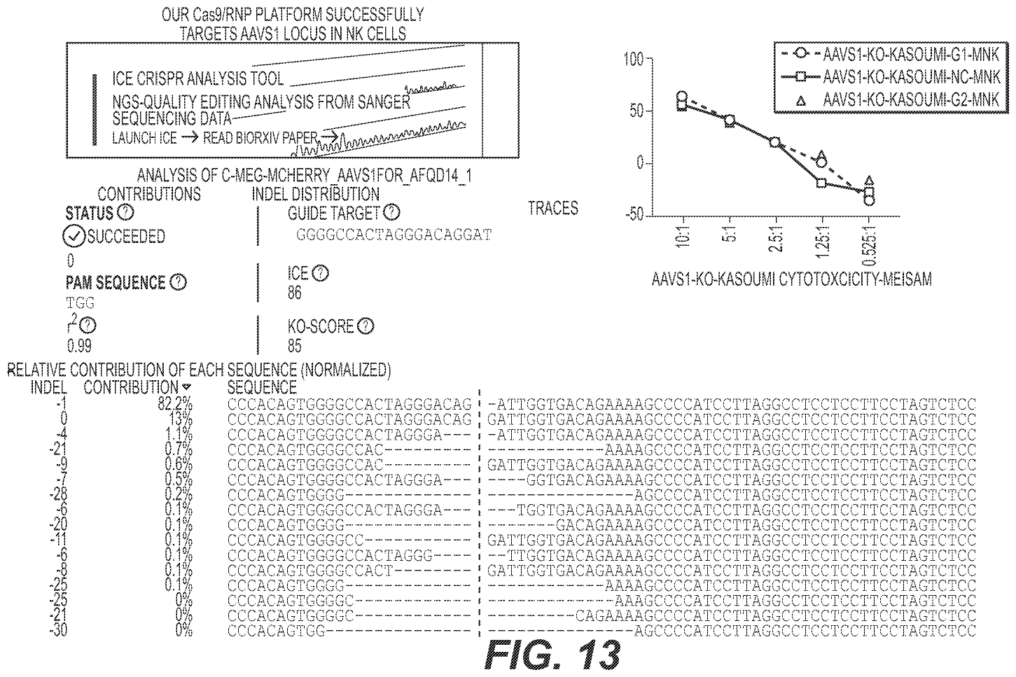

The ICE (Interference of CRISPR Edits) showed high efficiency of targeting the gene of interest using Cas9/RNP. Targeting AAVS1 does not change the Cytotoxicity effect of primary NK cells (FIG. 13).

(SEQ ID NO: 9)
gRNA used: GGGGCCACTAGGGACAGGAT

### 5. Example 5: Generating mCherry Positive Primary NK Cells as a Proof of Concept for CAR-NK Production Using Cas9/RNP Donors

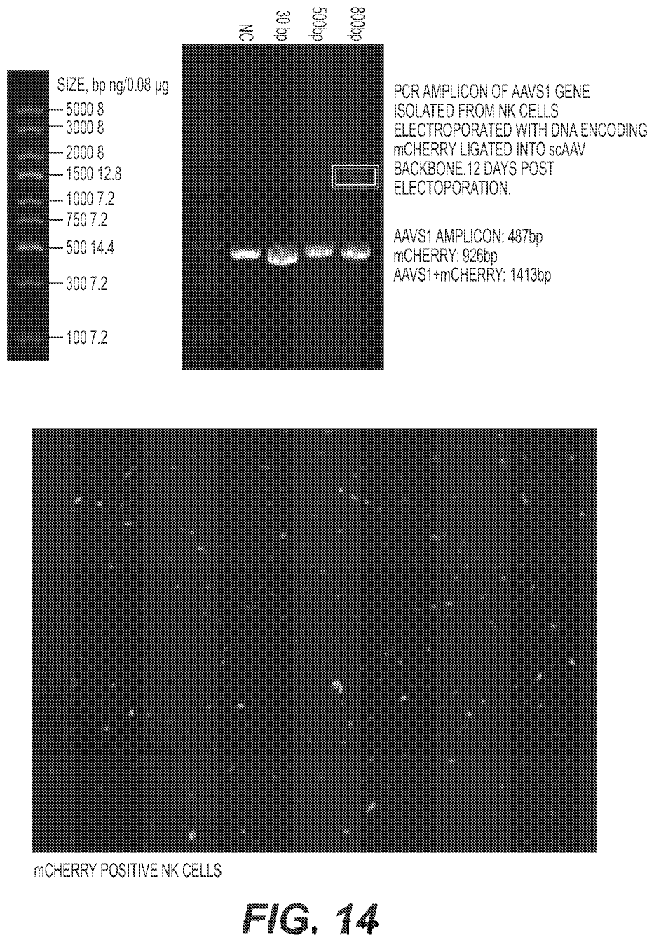

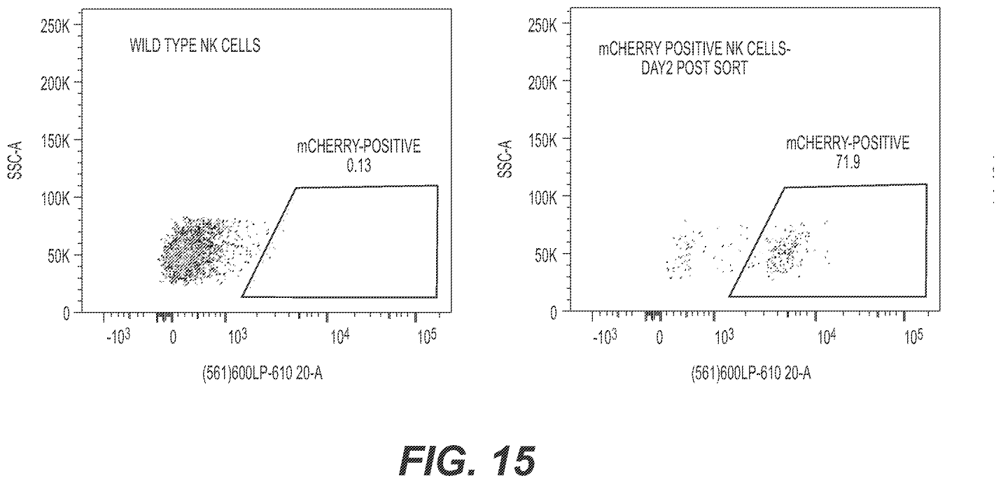

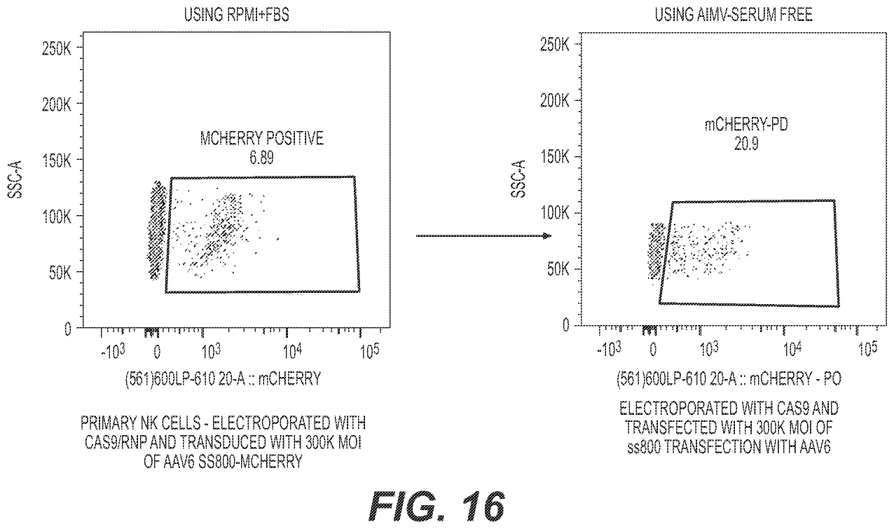

mCherry expressing human primary NK cells were generated using this approach. Furthermore, these modified primary NK cells were expanded by stimulating with irradiated mbIL21-expressing feeder cells and demonstrate the stable expression of a reporter gene (FIGS. 14, 15, and 16). This confirms the generation of primary and expanded CAR-NK cells.

(SEQ ID NO: 9)
gRNA used: GGGGCCACTAGGGACAGGAT

### 6. Example 6: Testing the Off-Target Effects

To Identify the off-target effects after using Cas9/RNP in NK cells, whole genome sequencing of the normal and modified NK cells (CD38-KO) was performed. The WGS showed no or very low (2 genes) off-targets based on the algorithm used for the predicting the candidate genes.

The list of the off-target candidate genes generated by Benching.com was investigated for the gRNA used for targeting CD-38. (5′-CTGAACTCGCAGTTGGCCAT-3′ (SEQ ID NO: 11)) and did not reveal any off-target. (The list of the off-target candidate genes as shown in Table 6)

TABLE 6

| | | | | | | | | On- | |
|---|---|---|---|---|---|---|---|---|---|
| | | | | Chromo- | | | Mis- | tar- | |
| Sequence | PAM | Score | Gene | some | Strand | Position | matches | get | |
| CTGAACTCGCAGTTGGCCAT | AGG | 100 | ENSG00000004468 | Chr4 | −1 | 15778418 | 0 | TRUE | gRNA |
| (SEQ ID NO: 12) | | | | | | | | | used to |
| | | | | | | | | | target |
| | | | | | | | | | CD38 |
| CTGTGCGTGCAGTTGGCCAT | CAG | 0.946765564 | | chr12 | −1 | 67239819 | 4 | FALSE | |
| (SEQ ID NO: 13) | | | | | | | | | |
| GTTAACTTACAGTTGGCCAT | AGG | 0.945468667 | | chr7 | −1 | 46392546 | 4 | FALSE | |
| (SEQ ID NO: 14) | | | | | | | | | |
| TGGACCTCTCAGTTGGCCAT | AGG | 0.942288961 | | chr1 | −1 | 115195756 | 4 | FALSE | |
| (SEQ ID NO: 15) | | | | | | | | | |
| CTGAACACTGAGTTGGCCAT | GGG | 0.932638593 | | chr4 | 1 | 135974807 | 3 | FALSE | |
| (SEQ ID NO: 16) | | | | | | | | | |
| TTGAACTTGTAGTTGGCCAA | AAG | 0.819283271 | | chr4 | −1 | 187686881 | 4 | FALSE | |
| (SEQ ID NO: 17) | | | | | | | | | |
| CCGACCTGGCAGTTGGCCCT | GGG | 0.634004237 | ENSG00000115649 | chr2 | −1 | 219173381 | 4 | FALSE | |
| (SEQ ID NO: 18) | | | | | | | | | |
| CCCACCTCGCAGGTGGCCAT | CGG | 0.6322725 | | chr20 | 1 | 61651638 | 4 | FALSE | |
| (SEQ ID NO: 19) | | | | | | | | | |
| CCCACCTCGCAGGTGGCCAT | CGG | 0.6322725 | | chr20 | 1 | 61648949 | 4 | FALSE | |
| (SEQ ID NO: 20) | | | | | | | | | |
| CTCATCTGGCAGTTGGCCTT | GGG | 0.604632172 | | chr8 | −1 | 22092249 | 4 | FALSE | |
| (SEQ ID NO: 21) | | | | | | | | | |
| CCTAACTCCCAGTTGGCCAG | TGG | 0.542406779 | | chr1 | −1 | 175788873 | 4 | FALSE | |
| (SEQ ID NO: 22) | | | | | | | | | |
| CTGTCTTCTCAGTTGGCCAT | GGG | 0.520512228 | | chr10 | −1 | 99880660 | 4 | FALSE | |
| (SEQ ID NO: 23) | | | | | | | | | |
| CAGAAATGGCAGTTGGCCAG | GAG | 0.519517522 | | chr12 | −1 | 119095636 | 4 | FALSE | |
| (SEQ ID NO: 24) | | | | | | | | | |
| ATGGACTCACATTTGGCCAT | CAG | 0.512406818 | ENSG00000174720 | chr4 | −1 | 112644556 | 4 | FALSE | |
| (SEQ ID NO: 25) | | | | | | | | | |
| CTTCACTCCCAGTTGGTCAT | TGG | 0.432613792 | | chr13 | −1 | 39496822 | 4 | FALSE | |
| (SEQ ID NO: 26) | | | | | | | | | |
| CTGGACTCCTATTTGGCCAT | AAG | 0.419713515 | | chrX | 1 | 6970537 | 4 | FALSE | |
| (SEQ ID NO: 27) | | | | | | | | | |
| CTCAACGTGCAGCTGGCCAT | GAG | 0.416350024 | ENSG00000100665 | chr14 | −1 | 94563541 | 4 | FALSE | |
| (SEQ ID NO: 28) | | | | | | | | | |
| TTGGACTCGCTGTTGGCCTT | GGG | 0.382094517 | ENSG00000170291 | chr17 | 1 | 7252571 | 4 | FALSE | |
| (SEQ ID NO: 29) | | | | | | | | | |
| TTGAACTGGCTGGTGGCCAT | CAG | 0.369648098 | | chr8 | 1 | 118209611 | 4 | FALSE | |
| (SEQ ID NO: 30) | | | | | | | | | |
| CTGACCTGTCAGCTGGCCAT | GGG | 0.35845875 | | chr6 | −1 | 170505358 | 4 | FALSE | |
| (SEQ ID NO: 31) | | | | | | | | | |
| CAGAACCCACAGTTGGCCAC | AGG | 0.358349447 | | chr10 | −1 | 31315629 | 4 | FALSE | |
| (SEQ ID NO: 32) | | | | | | | | | |
| CTGATGTCGCAGTTGTCCAT | GGG | 0.334541455 | | chr10 | 1 | 14397447 | 3 | FALSE | |
| (SEQ ID NO: 33) | | | | | | | | | |
| GTGAAGTCTCAGTTGGACAT | AGG | 0.277051878 | | chr5 | 1 | 66748005 | 4 | FALSE | |
| (SEQ ID NO: 34) | | | | | | | | | |
| CTGAGCTGGCAGATGGACAT | CAG | 0.261475216 | | chr19 | 1 | 30385960 | 4 | FALSE | |
| (SEQ ID NO: 35) | | | | | | | | | |
| CTGAACTGGAAGGTGGCCAG | GAG | 0.255794486 | | chr6 | −1 | 45577144 | 4 | FALSE | |
| (SEQ ID NO: 36) | | | | | | | | | |
| CTGAACTTGGAGCTGGCCAA | GGG | 0.255794486 | | chr10 | −1 | 48635391 | 4 | FALSE | |
| (SEQ ID NO: 37) | | | | | | | | | |
| CTGAAGTCGAGGTTGGCCAC | AAG | 0.230858484 | | chr20 | −1 | 1301156 | 4 | FALSE | |
| (SEQ ID NO: 38) | | | | | | | | | |
| CTGACGTCCCAGGTGGCCAT | GAG | 0.220622826 | | chr12 | −1 | 8382561 | 4 | FALSE | |
| (SEQ ID NO: 39) | | | | | | | | | |
| CTGCAATCACAGTTGGCCCT | AGG | 0.219482656 | | chr6 | −1 | 169035648 | 4 | FALSE | |
| (SEQ ID NO: 40) | | | | | | | | | |
| CAGAACATGCAGTTGTCCAT | GAG | 0.210305653 | | chr12 | −1 | 83067661 | 4 | FALSE | |
| (SEQ ID NO: 41) | | | | | | | | | |
| CTCAACTCCCTGGTGGCCAT | GGG | 0.208585768 | | chr18 | 1 | 45200028 | 4 | FALSE | |
| (SEQ ID NO: 42) | | | | | | | | | |
| CTGAAATGTCAGTTGGCCCT | GAG | 0.202352303 | | chr14 | −1 | 69318058 | 4 | FALSE | |
| (SEQ ID NO: 43) | | | | | | | | | |
| CTGAGCTCCCTGCTGGCCAT | GGG | 0.200652285 | | chr1 | 1 | 116380364 | 4 | FALSE | |
| (SEQ ID NO: 44) | | | | | | | | | |
| TTGAGCTCCCAGTTGCCCAT | AAG | 0.20020869 | | chr12 | 1 | 45093567 | 4 | FALSE | |
| (SEQ ID NO: 45) | | | | | | | | | |
| CTGAAATGGCTGATGGCCAT | AAG | 0.195328606 | | chr5 | 1 | 177246918 | 4 | FALSE | |
| (SEQ ID NO: 46) | | | | | | | | | |
| CTGAATGCACAGTTGGCCAA | TGG | 0.19038906 | | chr4 | −1 | 93982926 | 4 | FALSE | |
| (SEQ ID NO: 47) | | | | | | | | | |
| CTGACCTCCCAGATGGCCAC | CAG | 0.187845803 | | chr8 | −1 | 102685660 | 4 | FALSE | |
| (SEQ ID NO: 48) | | | | | | | | | |
| CTGAAATCCTAGTTGGCCCT | AGG | 0.186366471 | | chr1 | 1 | 240156657 | 4 | FALSE | |
| (SEQ ID NO: 49) | | | | | | | | | |
| CTGGACTCCCACATGGCCAT | CAG | 0.184200003 | | chr16 | −1 | 85987471 | 4 | FALSE | |
| (SEQ ID NO: 50) | | | | | | | | | |
| CTGATGTCGCACGTGGCCAT | GGG | 0.182391165 | | chr5 | −1 | 17200909 | 4 | FALSE | |
| (SEQ ID NO: 51) | | | | | | | | | |
| CTGAAACAGCAGTTAGCCAT | GAG | 0.172278175 | | chr3 | −1 | 67113967 | 4 | FALSE | |
| (SEQ ID NO: 52) | | | | | | | | | |
| CTGAAACCTCAGTTGGTCAT | CAG | 0.161062498 | | chr5 | 1 | 153440395 | 4 | FALSE | |
| (SEQ ID NO: 53) | | | | | | | | | |
| CTGAACTAGTGGTTGGCAAT | GAG | 0.160051112 | | chr1 | −1 | 234880279 | 4 | FALSE | |
| (SEQ ID NO: 54) | | | | | | | | | |
| CAGAACTAGCAGTTGGTAAT | AAG | 0.157208333 | | chr18 | 1 | 27784733 | 4 | FALSE | |
| (SEQ ID NO: 55) | | | | | | | | | |
| CTCAGCTCACAGTGGGCCAT | GAG | 0.151557283 | | chr14 | −1 | 101251958 | 4 | FALSE | |
| (SEQ ID NO: 56) | | | | | | | | | |
| CTCAAATGGCAGTGGGCCAT | CAG | 0.147277014 | | chr5 | 1 | 78106566 | 4 | FALSE | |
| (SEQ ID NO: 57) | | | | | | | | | |
| CTGAACTGTGAGTTGTCCAT | GAG | 0.146729115 | | chr22 | 1 | 27483121 | 4 | FALSE | |
| (SEQ ID NO: 58) | | | | | | | | | |
| CTGAACTGGAACTTGGCGAT | AGG | 0.143167158 | | chr1 | 1 | 204589932 | 4 | FALSE | |
| (SEQ ID NO: 59) | | | | | | | | | |
| CTGAACTCCCAGTGGGCCAC | TGG | 0.122047715 | | chr20 | −1 | 34536827 | 3 | FALSE | |
| (SEQ ID NO: 60) | | | | | | | | | |
| CTGAACTTGAATTTGCCCAT | GAG | 0.119165937 | ENSG00000175984 | chr1 | 1 | 114583481 | 4 | FALSE | |
| (SEQ ID NO: 61) | | | | | | | | | |

−Off-Target analysis:

### D. REFERENCES

Alici, E., Sutlu, T. & Sirac Dilber, M. Retroviral gene transfer into primary human natural killer cells. Methods Mol Biol. 506 127-137, doi:10.1007/978-1-59745-409-4_10, (2009).
Carlsten, M. & Childs, R. W. Genetic Manipulation of NK Cells for Cancer Immunotherapy: Techniques and Clinical Implications. Front Immunol. 6 266, doi:10.3389/fimmu.2015.00266, (2015).
Chmielecki, J. et al. Genomic Profiling of a Large Set of Diverse Pediatric Cancers Identifies Known and Novel Mutations across Tumor Spectra. Cancer Res. 77 (2), 509-519, doi:10.1158/0008-5472.can-16-1106, (2017).
DeWitt, M. A., Corn, J. E. & Carroll, D. Genome editing via delivery of Cas9 ribonucleoprotein. Methods. 121-122 9-15, (2017).
Eyquem, J. et al. Targeting a CAR to the TRAC locus with CRISPR/Cas9 enhances tumour rejection. Nature. 543 (7643), 113-117, doi:10.1038/nature21405, (2017).
Guven, H. et al. Efficient gene transfer into primary human natural killer cells by retroviral transduction. Exp Hematol. 33 (11), 1320-1328, doi:10.1016/j.exphem.2005.07.006, (2005).
Kim, S., Kim, D., Cho, S. W., Kim, J. & Kim, J. S. Highly efficient RNA-guided genome editing in human cells via delivery of purified Cas9 ribonucleoproteins. Genome Res. 24 (6), 1012-1019, doi:10.1101/gr.171322.113, (2014).
Lee, D. A., Verneris, M. R. & Campana, D. Acquisition, preparation, and functional assessment of human NK cells for adoptive immunotherapy. Methods Mol Biol. 651 61-77, doi:10.1007/978-1-60761-786-0_4, (2010).
Liang, X. et al. Rapid and highly efficient mammalian cell engineering via Cas9 protein transfection. J Biotechnol. 208 44-53, doi:10.1016/j.jbiotec.2015.04.024, (2015).
Liang, Z. et al. Efficient DNA-free genome editing of bread wheat using CRISPR/Cas9 ribonucleoprotein complexes. Nat Commun. 8 14261, doi:10.1038/ncomms14261, (2017).
Mehta, R. S. & Rezvani, K. Chimeric Antigen Receptor Expressing Natural Killer Cells for the Immunotherapy of Cancer. Front Immunol. 9 283, doi:10.3389/fimmu.2018.00283, (2018).
Rezvani, K., Rouce, R., Liu, E. & Shpall, E. Engineering Natural Killer Cells for Cancer Immunotherapy. Mol Ther. 25 (8), 1769-1781, doi:10.1016/j.ymthe.2017.06.012, (2017).
Schultz, L. M., Majzner, R., Davis, K. L. & Mackall, C. New developments in immunotherapy for pediatric solid tumors. Curr Opin Pediatr. 30 (1), 30-39, doi:10.1097/mop.0000000000000564, (2018).
Somanchi, S. S., Senyukov, V. V., Denman, C. J. & Lee, D. A. Expansion, purification, and functional assessment of human peripheral blood NK cells. J Vis Exp. (48), doi:10.3791/2540, (2011).
Sutlu, T. et al. Inhibition of intracellular antiviral defense mechanisms augments lentiviral transduction of human natural killer cells: implications for gene therapy. Hum Gene Ther. 23 (10), 1090-1100, doi:10.1089/hum.2012.080, (2012).
Viel, S. et al. TGF-beta inhibits the activation and functions of NK cells by repressing the mTOR pathway. Sci Signal. 9 (415), ra19, doi:10.1126/scisignal.aad1884, (2016).
Vouillot, L., Thelie, A. & Pollet, N. Comparison of T7E1 and surveyor mismatch cleavage assays to detect mutations triggered by engineered nucleases. G3 (Bethesda). 5 (3), 407-415, doi:10.1534/g3.114.015834, (2015).
Wang, M. et al. Efficient delivery of genome-editing proteins using bioreducible lipid nanoparticles. Proc Natl Acad Sci USA. 113 (11), 2868-2873, doi:10.1073/pnas.1520244113, (2016).
Zuris, J. A. et al. Cationic lipid-mediated delivery of proteins enables efficient protein-based genome editing in vitro and in vivo. Nat Biotechnol. 33 (1), 73-80, doi:10.1038/nbt.3081, (2015).

What is claimed is:

1. A method of genetically modifying an NK cell comprising
a) obtaining guide RNA (gRNA) specific for the target DNA sequence; and
b) introducing via electroporation into a target NK cell, a ribonucleoprotein (RNP) complex comprising a class 2 CRISPR/Cas endonuclease (Cas9) complexed with a corresponding CRISPR/Cas guide RNA that hybridizes to a target sequence within the genomic DNA of the NK cell.

2. The method of claim 1, wherein the genome of the NK cell is modified by insertion or deletion of one or more base pairs, by insertion of a heterologous DNA fragment (e.g., the donor polynucleotide), by deletion of an endogenous DNA fragment, by inversion or translocation of an endogenous DNA fragment, or a combination thereof.

3. The method of claim 1, wherein the NK cells are primary or expanded NK cells.

4. The method of claim 3, wherein the primary NK cells are incubated for 2, 3, or 4 days in the presence of IL-2 prior to electroporation.

5. The method of claim 3, wherein the primary NK cells are expanded for 4 days in the presence of irradiated feeder cells prior to electroporation.

6. The method of claim 1, further comprising expanding the modified NK cells with irradiated mbIL-21 expressing feeder cells following electroporation.

7. The method of claim 1, wherein the method further comprises forming the RNP complex by diluting 36 μM cas9 into a solution of 200 μM crRNA and TracerRNA.

8. An NK cell modified by the method of claim 1.

9. A genetically modified NK cell comprising a knockout of the gene encoding the transforming growth factor-β receptor 2 (TGFBR2) or hypoxanthine phosphoribosyltransferase 1 (HPRT1).

10. A method of adoptively transferring an engineered NK cells to a subject in need thereof said method comprising
a) obtaining a target NK cell to be modified;
b) obtaining gRNA specific for the target DNA sequence;
c) introducing via electroporation into the target NK cell, a RNP complex comprising a class 2 CRISPR/Cas endonuclease (Cas9) complexed with a corresponding CRISPR/Cas guide RNA that hybridizes to a target sequence within the genomic DNA of the target NK cell creating an engineered NK cell; and
d) transferring the engineered NK cell into the subject.

11. The method of claim 10, wherein the subject has a cancer.

12. The method of claim 10, wherein the NK cell is a primary NK cell that has been modified ex vivo and after modification transferred to the subject.

13. The method of claim 10, wherein the NK cell is an autologous NK cell.

14. The method of claim 10, wherein the NK cell is from an allogeneic donor source.

15. The method of claim 10, wherein the NK cell is expanded with irradiated mbIL-21 expressing feeder cells prior to administration to the subject.

16. The method of claim 10, wherein the NK cell is expanded in the subject following transfer of the NK cells to the subject via the administration of IL-21 or irradiated mbIL-21 expressing feeder cells.

17. The method of claim 10, wherein the RNP complex targets the TGFRB2 or HPRT1 gene.

18. The A method of treating a cancer in a subject comprising administering to the subject an NK cell that has been modified to comprise a knockout of the TGFBR2 gene.
